(19) United States
(12) Patent Application Publication
(10) Pub. No.: US 2024/0108591 A1
(43) Pub. Date: Apr. 4, 2024

(54) OR2L13 AGONISTS TO TREAT CVD WITH DYSREGULATED PLATELET ACTIVATION

(71) Applicant: The Cleveland Clinic Foundation, Cleveland, OH (US)

(72) Inventors: Shaun Stauffer, Brentwood, TN (US); Scott Cameron, Solon, OH (US); Vara Prasad Josyula, Beachwood, OH (US)

(21) Appl. No.: 18/352,450

(22) Filed: Jul. 14, 2023

Related U.S. Application Data
(60) Provisional application No. 63/389,064, filed on Jul. 14, 2022.

(51) Int. Cl. A61K 31/122; A61K 31/05; A61P 7/02; A61P 9/00

(52) U.S. Cl. CPC A61K 31/122; A61K 31/05; A61P 7/02; A61P 9/00

(57) ABSTRACT

Provided herein are compositions, systems, and methods for treating a subject with, or elevated risk for, a cardiovascular disease (CVD) involving dysregulated platelet activation comprising: treating the subject with a composition comprising an OR2L13 activating agent (e.g., carvone; 2-tert-Butyl-4-Methylphenol; or 2-tert-Butyl-5-Methylphenol). In certain embodiments, the CVD comprises abdominal aortic aneurysm (AAA) or thrombosis.

### CROSS-REFERENCE TO RELATED APPLICATIONS

This application claims the benefit of U.S. Provisional Application No. 63/389,034, filed Jul. 14, 2022, the content of which is herein incorporated by reference in its entirety

### FIELD OF THE INVENTION

Provided herein are compositions, systems, and methods for treating a subject with, or elevated risk for, a cardiovascular disease (CVD) involving dysregulated platelet activation comprising: treating the subject with a composition comprising an OR2L13 activating agent (e.g., carvone; 2-tert-Butyl-4-Methylphenol, or 2-tert-Butyl-5-Methylphenol). In certain embodiments, the CVD comprises abdominal aortic aneurysm (AAA) or thrombosis.

### SEQUENCE LISTING STATEMENT

The contents of the electronic sequence listing titled 39231-202_SEQUENCE_LISTING.xml (Size: 4,666 bytes; and Date of Creation: Jul. 14, 2023) is herein incorporated by reference in its entirety.

### BACKGROUND

Remodeling of the infrarenal aorta that results in AAA affects millions of individuals and carries an extraordinarily high out of hospital mortality rate if this pathological process progresses to aortic rupture (1). Most individuals with AAA are asymptomatic but harbor common risk factors, including male sex, tobacco use, advanced age, and atherosclerosis. Factors accelerating AAA growth involve mechanical forces on the blood vessel wall, and enzymatic degradation of the extracellular matrix (ECM) (2).

As blood transitions from steady laminar flow (S-flow) to disturbed flow (D-flow) in aneurysmal arteries, circulating platelets may become mechanically activated (11). Mechanical platelet activation in patients with aneurysmal arteries was postulated using computational fluid dynamic modeling. Agonist-mediated platelet activation has never been proven in humans with infrarenal AAA or in relevant animal models of AAA (6). Subjecting blood to mechanical forces promotes the development of luminal thrombus. Luminal thrombus was suggested to accelerate aneurysmal growth of arteries (12, 13). In a recent randomized clinical trial in patients with small AAA, treatment with the platelet P2Y12 receptor antagonist ticagrelor did not alter AAA growth indicating a more mechanistic approach to understanding platelet activation in AAA is required (14). Presently-available therapeutic agents to abrogate platelet reactivity focus on cell surface receptors initiating biochemical second messenger-mediated signaling cascades. There are no therapeutic agents to inhibit biomechanical platelet activation

### SUMMARY

Provided herein are compositions, systems, and methods for treating a subject with, or elevated risk for, a cardiovascular disease (CVD) involving dysregulated platelet activation comprising: treating the subject with a composition comprising an OR2L13 activating agent (e.g., carvone; 2-tert-Butyl-4-Methylphenol; or 2-tert-Butyl-5-Methylphenol). In certain embodiments, the CVD comprises abdominal aortic aneurysm (AAA) or thrombosis.

In some embodiments, provided herein are methods of treating a subject with, or elevated risk for, a cardiovascular disease (CVD) involving dysregulated platelet activation comprising: treating a subject with a composition comprising an OR2L13 activating agent; wherein the subject has, or is at elevated risk for, a CVD involving dysregulated platelet activation (e.g., resulting in thrombosis that is apparent in thrombotic disorders including and beyond myocardial infarction and stroke).

In particular embodiments, provided herein are compositions comprising: a) a blood or plasma sample from a subject that has, or is at elevated risk for, a CVD involving dysregulated platelet activation; and b) a composition comprising an OR2L13 activating agent.

In other embodiments, provided herein are methods of screening a candidate compound comprising: a) contacting a sample with a candidate OR2L13 activating agent, wherein the sample comprises activated platelets, and b) determining if the candidate OR2L13 activating agent reduces activation of the activated platelets. In some embodiments, the sample comprises a blood or plasma sample. In other embodiments, the blood or plasma sample is from a subject that has, or is at elevated risk for, a CVD involving dysregulated platelet activation.

In particular embodiments, the OR2L13 activating agent binds OR2L13 on platelets in the subject. In certain embodiments, the OR2L13 activating agent reduces platelet activation in the subject. In other embodiments, the CVD comprises abdominal aortic aneurysm (AAA). In additional embodiments, the CVD comprises thrombosis. In some embodiments, the CVD is selected from the group consisting of: myocardial infarction, stroke, coronary artery disease, angina, heart failure, heart valve disease, and heart attack.

In certain embodiments, the OR2L13 activating agent comprises Carvone. In some embodiments, the Carvone comprises (−) Carvone or (+) carvone or a derivative with the same core carvone structure, but additional atoms added thereto. In particular embodiments, the OR2L13 activating agent is selected from the group consisting of: 2-tert-Butyl-5-Methylphenol; 2-tert-Butyl-4-Methylphenol; (−)-Carvone; 2-Aminomethyl-5-tert-butylphenol; 2,5-Methylphenol; 2-Methyl-5-(Propan-2-yl) Benzene-1,4-Diol; 5-ChloroCarvacrol; Carvacrol; 5-tert-Butyl-2-Methyl Phenol; and Irone.

In particular embodiments, the subject is a human. In certain embodiments, the treating comprises administering the composition to the subject orally or via an aerosol. In some embodiments, the treating comprises administering the composition to the subject intravenously. In other embodiments, the treating comprises providing the composition to the subject such that they administer the composition to themselves (e.g., orally, by an inhaler, or intravenously).

In additional embodiments, the methods further comprise, prior to the treating, the step of testing a blood or plasma sample from the subject to determine if platelets in the sample are activated. In other embodiments, the methods further comprise, after the treating, the step of testing a blood or plasma sample from the subject to determine if platelets in the sample are activated.

In certain embodiments, the subject has a CVD involving dysregulated platelet activation. In additional embodiments, the subject is at elevated risk for a CVD involving dysregulated platelet activation (e.g., diagnosed as having an elevated risk for a CVD involving dysregulated platelet activation).

### BRIEF DESCIPTION OF THE DRAWINGS

The patent or application file contains at least one drawing executed in color. Copies of this patent or patent application publication with color drawings will be provided by the Office upon request and payment of the necessary fee.

FIG. 1. Platelet reactivity is enhanced in patients with AAA. A. Washed platelets from patients with AAA (n=18) compared to healthy subjects (n=10). Platelet activation before and after stimulation with a thromboxane receptor agonist (U46619) or a PAR1 agonist (TRAP-6) for minutes. Platelet activation quantified by FACS as median fluorescence intensity (MFI) and represented as median (horizontal line) in a box and whisker plot for each group performed in quadruplicate and summed for each patient at each concentration of agonist. *P<0.05 and **P<0.01 vs. healthy control. Group differences were analyzed by the Kruskal-Wallis test followed by Dunn's post test correction. B. Human platelet RNA was extracted from healthy subjects (n=7) and compared to patients with AAA (n=6) by mRNA-sequencing. Volcano plot shows genes upregulated (red) and downregulated (green) in patients with AAA. Dashed line is the threshold of discrimination. Heatmap with upstream Olfactory Receptor 2L13 (OR2L13) and downstream anoctamin are increased in AAA platelets compared to heathy conditions. C. RNA isolated from washed platelets followed by CD45-mediated immunodepletion of white blood cells, then qRT-PCR normalized to the platelet GPIIb gene (ITGA2B). Data from 4 individual healthy males, each run in quadruplicate, is indicated by violin plots for the three olfactory receptors present in every subject tested. D. Lysate from healthy human platelets or human brain (positive control) separated by SDS-PAGE before probing with an anti-OR2L13 antibody. E. Platelets from healthy humans on a fibrinogen matrix assessed for OR2L13 immunofluorescence by confocal microscopy. A FITC-tagged OR2L13 antibody (green) and rhodamine-tagged phalloidin (red) for filamentous actin. DIC=Differential Interference Contrast. Yellow bar =10 μm.

FIG. 2. Platelet olfactory receptor expression increases in AAA. A. Alpha granules were identified by a rhodamine-tagged p-selectin antibody (red). Dense granules were detected by staining phosphate groups in adenosine diphosphate (ADP, blue) by confocal microscopy. Spearman's rho was determined by computer-generated co-localization overlay of OR2L13 and p-selectin (0.71±0.04) or ADP (0.25±0.07) and represented ad mean±SEM, P<0.0001 by Student's t-test, n=7-8 individual subjects. Red Bar=10 μm. B. Immunoblotting platelets for OR2L13 expression which is increased in AAA compared to healthy conditions and represented as mean±SEM by Student's t-test, P=0.035, n=4 in each group. C. OR2L13 localization and surface area by confocal microscopy (left graph) and flow cytometry (right graph). Confocal microscopy was used to visualize platelet surface area by spreading on a fibrinogen matrix (right) and quantified as mean surface area±SEM, n=7-10, Mann Whitney U. Red Bar=5μm. Platelet surface OR2L13 quantified by FACS as mean fluorescence intensity (MFI) ±SEM, n=4-30 6, Mann Whitney U.

FIG. 3. Bomechanical stimulation of platelets increases activation. A. Color flow Doppler imaging of a large infrarenal AAA demonstrates alternating direction of blood flow (Disturbed Flow, D-Flow). B. An in vitro flow and cone system was used to subject healthy platelets to steady laminar flow (S-flow) or disturbed flow (D-flow) for 120 mins. C. Platelet activation following S-Flow and D-flow quantified by surface p-selectin expression or fibrinogen binding by FACS as mean fluorescence intensity (MFI)±SEM, n=4, P<0.001 vs. static flow by ANOVA, followed by Bonferroni correction. D. Healthy human platelets subjected to static flow, steady laminar flow (S-flow) or disturbed flow (D-flow) for 120 mins. Immobilization of platelets after S-flow and D-flow on a fibrinogen matrix and visualization by confocal microscopy. Platelets were stained with a FITC-tagged antibody for OR2L13 (green) and rhodamine-tagged phalloidin for actin (red). Yellow bar=5 μm. E. In a separate set of experiments platelet membrane OR2L13 was quantified after S-flow and D-flow by FACS as mean fluorescence intensity (MFI)±SEM, n=4, P<0.001 vs. static flow by ANOVA, followed by Bonferroni correction.

FIG. 4. Characterization of OR2L13 agonists. A. Human OR2L13 cloned in frame with HA (GFP in a secondary cassette, green) and co-expressed with Receptor Transport Protein ls (RTP1s) (mCherry in a secondary cassette, red) stably in HEK293/cAMP cells. Microscopy (left) and Western blotting (right) confirm OR2L13-HA expression. IP=immunoprecipitation. TCL=total cell lysate. OR2L13 ligands stimulate (G_{olf}) and adenyl cyclase produces cAMP B. A cAMP-Response Element (CRE) expressing HEK293 cells with stable integration of OR2L13-HA was utilized to screen for ligands. C. Alpha screen of olfactory ligands in OR2L13 transduced/non-transduced cells with >1.0 ratio (red dashed line) for OR2L13 activation and (−) Carvone activates OR2L13 to generate cAMP (performed in duplicate, horizontal line is mean). Confirmatory experiment with vehicle vs. (−) carvone is shown as mean±SEM, n=4, bottom. * P=0.079, **P=0.0001 and ***P<0.0001, vs. vehicle by ANOVA followed by Bonferroni correction.

FIG. 5. A conserved olfactory receptor signal transduction pathway in healthy platelets. A. Proposed platelet OR2L13 signal transduction. Levo-carvone (L-Carvone) binds to OR2L13 on platelets to generate cAMP through adenylyl cyclase activation, and cAMP changes the cytosolic concentration of calcium (Ca 2+) directly and chloride conductance (C1⁻) while inhibiting platelet reactivity through known, well-described mechanisms involving protein kinase (PKA). B. (−) Carvone (300 μM) or forskolin (10 μM) incubation with human platelets for 5 minutes generates cAMP. Forskolin is a positive control for adenylyl cyclase activity and ATP hydrolysis to cAMP, *P=0.0059, **P=0.0089 vs. vehicle by Kruskal—Wallis, followed by Dunn post-test n=7. C. Carvone stimulation (300 μM) of healthy platelets promotes chloride efflux. Data are mean±SEM (n=4, MQAE fluorescence). *P=0.0134 (−) carvone vs control by student's t-test. D. Carvone stimulation (300 μM) of healthy platelets promotes local brief calcium transients 20-80% above baseline is sustained compared with thrombin which attenuates over time (n=5 human platelets, Fura-2 fluorescence). MQAE=-[ethoxycarbonylmethyl]-6-methoxy-quinoliniumbromide. Arrowhead=drug addition.

FIG. 6. A OR2L13 agonist potently inhibits platelet aggregation. A. Platelets were isolated from healthy humans and preincubated with (−) carvone or (+) carvone (300 μM) for 30 minutes. Platelets were stimulated with ADP (0.1 μM) and light transmission aggregometry assessed platelet activation. Representative tracings for each agonist are shown. B. Carvone (300 μM) or forskolin (10 μM) incubation individually or together for 30 minutes followed by platelet stimulation with ADP (0.1 μM). Light transmission aggregometry assessed platelet activation. Representative aggregometry tracings are shown. V=vehicle, C=(−)carvone, F=forskolin. Differences between groups by one-way ANOVA followed by Bonferroni correction *P<0.0001, **P=0.0003, ***P<0.0001 vs. vehicle (n=3). C. Summary data from light transmission aggregometry for each agonist as mean±SEM, n=3, *P=0.08 vs. vehicle **P=0.0013 vs. vehicle by ANOVA followed by Bonferroni correction. D. Platelets isolated from healthy humans and preincubated with (−) carvone (0-500 μM) for 30 minutes then stimulated with ADP (0.1 μM). Light transmission aggregometry assessed platelet activation. Representative tracings for each agonist are shown. E. Summary data for each concentration of (−) carvone as mean±SEM by ANOVA in the presence of ADP stimulation (0.1 μM). The downward red arrow is ADP in the presence of vehicle to which all data points were compared. *P<0.05 vs. vehicle, n=3 in each group by ANOVA, followed by Bonferroni correction. The broken blue line indicates the log IC₅₀ concentration of (−) carvone. E. Summary data for each concentration of carvone derivatives and closely-related carvone compounds as mean±SEM by ANOVA in the presence of ADP stimulation (0.1 μM). The downward red arrow is ADP in the presence of vehicle to which all data points were compared. *P<0.05 vs. vehicle, n=3 in each group by ANOVA, followed by Bonferroni correction. The broken blue line indicates the log IC50 concentration of (−) carvone.

FIG. 7. Biomechanical platelet activation is inhibited by OR2L13 agonists. A. In vitro exposure of healthy platelets to steady laminar flow (S-flow) or disturbed flow (D-flow) for 60 mins after 30 minute pre-treatment with vehicle or (−) Carvone (300 μM). Platelet activation quantified by surface p-selectin expression as mean fluorescence intensity (MFI)±SEM, n=3 in each group by 2-way ANOVA. B. Calcein green-loaded healthy human blood flowing through a collagen-I coated microfluidics chamber at 40 dyn/cm² and imaged by confocal microscopy for adherent thrombus (green puncta) at three minutes. Data are represented as mean thrombus area of random fields±SEM, n=6-7 in each group following 30 minutes of vehicle (0.25% DMSO) or (−) carvone treatment (300 μM). Differences between groups was assessed by the student's t-test, * P<0.0001 vs. vehicle. C. WT Fvb/tac mice were given 100 mg/Kg/day (−) carvone IP for 3 days. Platelets were isolated and stimulated in the presence of thrombin. Platelet activation quantified by FACS as mean fluorescence intensity (MFI)±SEM, n=4, *P=0.061 or **P=0.007 and ***P<0.0001. vs. vehicle by ANOVA, followed by Bonferroni correction. D. Time to hemostasis in mice treated with (−) carvone following surgical amputation of tail tip in seconds±SEM, n=6-9 in each group, *P=0.0046 vs. vehicle by Mann Whitney U.

FIG. 8. Platelets are biomechanically activated in mice and humans with AAA. A. WT male C57BL/6J mice treated with topical aortic elastase or heat inactivated elastase (sham) with BAPN in the drinking water developed stable AAA with luminal thrombus (yellow arrow). Top: B-mode ultrasound, Middle color spectral Doppler interrogation of the aneurysmal region shows D-flow (Doppler (red <>blue). Bottom: Dissecting video microscopy at the end of the protocol with marked aneurysm (white arrow) below the renal artery. Time point is 6 weeks post-AAA. B. Aortic diameter by ultrasound is shown as mean±SEM (diameter indicated below graph), 20 *P=0.114, **P=0.0046 by repeated measures ANOVA, followed by Bonferroni correction, n=5. C. Platelet surface OR2L13 expression for translocation from baseline and after 4 weeks of AAA induction as mean florescence intensity (MFI)±SEM, n=4 *P=0.0251 by ANOVA, followed by Bonferroni correction. D. Platelet surface p-selectin for platelet activation after dose-dependent thrombin stimulation in sham-operated or AAA mice weeks 1-4 post-AAA induction. Differences between groups determined by 2-way ANOVA. * P<0.01, ** P<0.001, n=4 in each group. E. An in vitro flow and cone system was used to subject healthy platelets to static flow (0) or disturbed flow (D-flow) for 0-90 mins. Solid bars=healthy subject and hatched bars=AAA patient. Platelet activation as mean surface p-selectin±SEM, n=3 in each group by ANOVA followed by Bonferroni correction.

FIG. 9. Platelet OR2L13 agonists suppress platelet reactivity and AAA growth. A. Non-reducing SDS-PAGE of human platelet lysates for MMP activity examined by in-gel zymography and protein content examined by immunoblotting. MMP9 content was similar though activated MMP is enriched in platelets from patients with AAA compared to healthy individuals (n=4-5) quantified as mean±SEM and normalized to GAPDH (n=4-10). Differences between groups was examined the student's t-test. MMP=Metalloproteinase. TIMP=Tissue Inhibitor of MMP. Protein size in indication in Kilodaltons (KDa). B. Aortic diameter by ultrasound following Aspirin (30 mg/L, drinking water) therapy or daily i.p. injection of 100 mg/Kg (−) carvone compared with vehicle starting at day 7 protects in fvb/tac mice from (n=4-20), *P=0.0002 and ** P<0.0001 vs. vehicle by ANOVA, followed by Bonferroni correction. C. Aortic lysate at week 4 following AAA was assessed for MMP activity (zymography). Actin and total protein are loading controls. Data are representative of 9 WT mice (n=3 in each group) at 4 weeks. *P=0.012 and **P=0.0002 vs. vehicle by 2-way ANOVA. Yellow asterisk (*) is a purified and activated MMP2 standard (S). Protein size is indicated in Kilodaltons (KDa).

FIG. 10. CRISPR-Cas9 disruption of the Olfr 168 locus augments platelet reactivity. A. Sequencing primer design spanning the OR2L13 (fvb/Tac murine olfr168) intron/exon boundaries. Genotyped pups after RNP injection show the edit (arrow) following PCR with #1/#4 primers and deletion following PCR with #1/#2 primers (right gel, last lane). B. Sanger sequencing shows an edit in the upstream region of the olfr168 locus (box). Amplified product size is indicated in kilobases (Kb). WT=wild-type. Het=heterozygote. KO=knock out (olfr168 ^{-/-}). C. Immunoblotting platelet lysate for the protein product of the wild type fvb/Tac (WT), and null (KO) murine alleles for the olfr 168 gene using an anti-OR2L13 antibody. D. Electron micrographic images of individual wild-type and olfr168^{-/-}mouse platelets with increased thrombotic granule content apparent in olfr168^{-/-}mouse platelets (arrowheads). Scale bar is 0.5 μm in the magnified image. E. Isolated olfr168^{-/-}platelets show increased reactivity when stimulated with thrombin ex vivo. Platelet activation is shown as mean surface p-selectin±SEM, n=4 in each group by ANOVA followed by Bonferroni correction, *P<0.0001.

FIG. 11. Olfr168-Deficient mice have enhanced platelet and aortic MMP2 activation with accelerated aortic aneurysm growth and rupture. A. B-mode ultrasound of infrarenal AAA at week 4 after elastase induction, and color Spectral Doppler showing marked D-flow in AAA. Bottom: color spectral Doppler of aortic aneurysmal segments showing disturbed blood in 30 olfr 168^{-/-}mice. B. Left: AAA growth by ultrasound for WT or olfr168^{-/-}fvb/tac mice (n=5-17), *P=0.0037 WT vs. olfr168^{-/-}by Kruskal—Wallis, followed by Dunn post-test. C. Kaplan-Meier survival curves for AAA rupture in WT or olfr168^{-/-}fvb/tac mice (n=10 in each group). Difference between groups was evaluated by the Log-rank (Mantel-Cox) test, P=0.042 between groups. D. Aortic lysate at 4 weeks following AAA assessed for MMP activity (zymography) in fvb/tac mice at 4 weeks (n=8 in each group), *P=0.028 vs. Olfr168^{-/-}by Mann-Whitney U test. GAPDH and total protein (stain) are loading controls. E. Platelet lysate at week 4 following 5 AAA was assessed for MMP2 activity (zymography) in fvb/tac mice at 4 weeks (n=4 in each group), *P=0.023 vs. Olfr168^{-/-}by student's t-test.

FIG. 12. Hypothetical model of Platelet-mediated MMP9 activity and AAA progression in D-flow environments. As blood moves from steady laminar flow (S-flow) to disturbed flow (D-flow) in aneurysmal or atherosclerotic stenotic aorta in humans, platelets are biomechanically activated, secreting matrix metalloproteinase 9 (M1V1P9) that further remodeling the aorta in aneurysmal disease.

FIG. 13 shows the molecular structures of: (−) carvone; (+) carvone; carvacrol; carveol; 5-ChloroCarvacrol; 5-tert-Butyl-2-Methyl Phenol; 2-tert-Butyl-4-Methylphenol; Irone; 2-Aminomethyl-5-tert-butylphenol; and 2-tert-Butyl-5-Methylphenol.

### DETAILED DESCRIPTION

Provided herein are compositions, systems, and methods for treating a subject with, or elevated risk for, a cardiovascular disease (CVD) involving dysregulated platelet activation comprising: treating the subject with a composition comprising an OR2L13 activating agent (e.g., carvone). In certain embodiments, the CVD comprises abdominal aortic aneurysm (AAA) or thrombosis.

The OR2L13 activating agents recited herein (e.g., (−) carvone) may be formulated in pharmaceutical formulations and/or medicaments. For example, for injection, the pharmaceutical formulation and/or medicament may be a powder suitable for reconstitution (e.g., at a hospital or pharmacy) with an appropriate solution (e.g., IV solution, such as Lactated Ringers solution). Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. In certain embodiments, the OR2L13 activating agents are mixed with an organic polar solvent. In certain embodiments, the OR2L13 activating agents are mixed with a buffer (e.g., phosphate buffered saline).

In certain embodiments, the pharmaceutical formulations (e.g., comprising carvone) are administered orally, in the form of a pill capsule, gel-cap, or the like. In some embodiments, the oral administration is 1-1500 mg of OR2L13 activating agents per kilogram of subject (e.g., 1 ... 10 . . . 75 . . . 100 . . . 125 . . . 150 . . . 200 . . . 250 . . . 300 . . . 400 . . . 500 . . . 650 . . . 800 . . . 1000 . . . 1500 mg/kg). In certain embodiments, provided herein are pills or capsules containing an OR2L—activating agent. In particular embodiments, the pills or capsules (e.g., softgels) have an enteric coating.

Dosage forms for the topical (including buccal and sublingual) or transdermal or oral administration of OR2L13 activating agents of the invention include powders, sprays, pills, gel-caps, ointments, pastes, creams, lotions, gels, solutions, and patches. The OR2L13 activating agent herein may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pill or capsule herein comprises a gelatin encapsulated dosage form (e.g., a softgel). In certain embodiments, the gelatin encapsulation of the agent herein is composed of gelatin, glycerin, water, and optionally caramel. In particular embodiments, the pills and capsules herein are coated with an enteric coating (e.g., to avoid the acid environment of the stomach, and release most of the agent in the small intestines of a subject). In some embodiments, the enteric coating comprises a polymer barrier that prevents its dissolution or disintegration in the gastric environment, thus allowing the OR2L13 activating agent herein to reach the small intestines. Examples of enteric coatings include, but are not limited to, Methyl acrylate-methacrylic acid copolymers; Cellulose acetate phthalate (CAP); Cellulose acetate succinate; Hydroxypropyl methyl cellulose phthalate; Hydroxypropyl methyl cellulose acetate succinate (hypromellose acetate succinate); Polyvinyl acetate phthalate (PVAP); Methyl methacrylate-methacrylic acid copolymers; Shellac; Cellulose acetate trimellitate; Sodium alginate; Zein; COLORCON, and an enteric coating aqueous solution (ethylcellulose, medium chain triglycerides [coconut], oleic acid, sodium alginate, stearic acid) (e.g., coated softgels). Additional enteric coatings are described in Hussan et al., IOSR Journal of Pharmacy, e-ISSN: 2250-3013, p-ISSN: 2319-4219, Volume 2 Issue 6, Nov-Dec. 2012, PP.05-11, herein incorporated by references in its entirety, and particularly for its description of enteric coatings.

The OR2L13 activating agents of the invention (e.g., carvone) may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. Formulations for inhalation administration contain as excipients, for example, lactose, polyoxyethylene-9-lauryl ether, glycocholate and deoxycholate. Aqueous and nonaqueous aerosols are typically used for delivery of the agents herein by inhalation.

Ordinarily, an aqueous aerosol is made by formulating an aqueous solution or suspension of the OR2L13 activating agents together with conventional pharmaceutically acceptable carriers and stabilizers. The carriers and stabilizers vary with the requirements of the particular compound, but typically include nonionic surfactants (e.g., TWEENs, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. A nonaqueous suspension (e.g., in a fluorocarbon propellant) can also be used to deliver the agents of the invention.

Aerosols containing OR2L13 activating agents for use according to the present invention are conveniently delivered using an inhaler, atomizer, pressurized pack or a nebulizer and a suitable propellant, e.g., without limitation, pressurized dichlorodifluoromethane, trichlorofluoromethane, dichlorotetrafluoroethane, nitrogen, air, or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the OR2L13 activating agents and a suitable powder base such as lactose or starch. Delivery of aerosols of the present invention using sonic nebulizers is advantageous because nebulizers minimize exposure of the agent to shear, which can result in degradation of the compound.

For nasal administration, the pharmaceutical formulations and medicaments with the OR2L13 activating agents may be a spray, nasal drops or aerosol containing an appropriate solvent(s) and optionally other compounds such as, but not limited to, stabilizers, antimicrobial agents, antioxidants, pH modifiers, surfactants, bioavailability modifiers and combinations of these. For administration in the form of nasal drops, the agent may be formulated in oily solutions or as a gel. For administration of nasal aerosol, any suitable propellant may be used including compressed air, nitrogen, carbon dioxide, or a hydrocarbon based low boiling solvent.

Transdermal patches may be employed herein (e.g., comprising carvone), and have the added advantage of providing controlled delivery of a compound of the invention to the body. Such dosage forms can be made by dissolving or dispersing the agent in the proper medium. Absorption enhancers can also be used to increase the flux of the agent across the skin. The rate of such flux can be controlled by either providing a rate controlling membrane or dispersing the compound in a polymer matrix or gel.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

Specific dosages of the OR2L13 activating agent herein may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

In certain embodiments, the OR2L13 activating agent herein are administered in a cycle of less than about 3 weeks, about once every two weeks, about once every 10 days or about once every week or once per day. One cycle can comprise the administration of an agent herein and optionally a second active agent (e.g., another anti-platelet agent) by infusion over about 90 minutes every cycle, about 1 hour every cycle, about 45 minutes every cycle, about 30 minutes every cycle or about 15 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In particular embodiments, courses of treatment can be administered concurrently to a subject, i.e., individual doses of the OR2L13 activating agent herein and secondary therapeutic are administered separately yet within a time interval such that the agent herein can work together with the additional therapeutic agent. For example, one component can be administered once per week in combination with the other components that can be administered once every two weeks or once every three weeks. In other words, the dosing regimens are carried out concurrently even if the therapeutics are not administered simultaneously or during the same day.

### EXAMPLES

### Example 1

### Platelet Olfactory Receptor Activation Limits Platelet Reactivity and Growth of Aortic Aneurysms

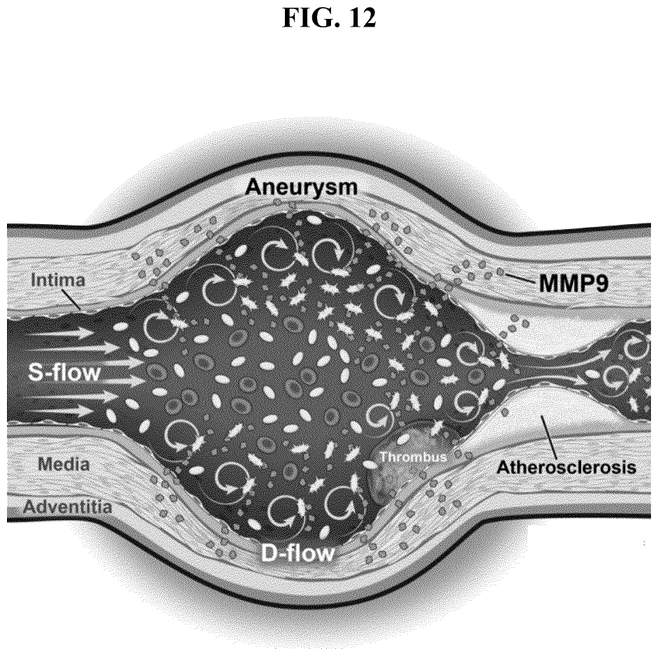

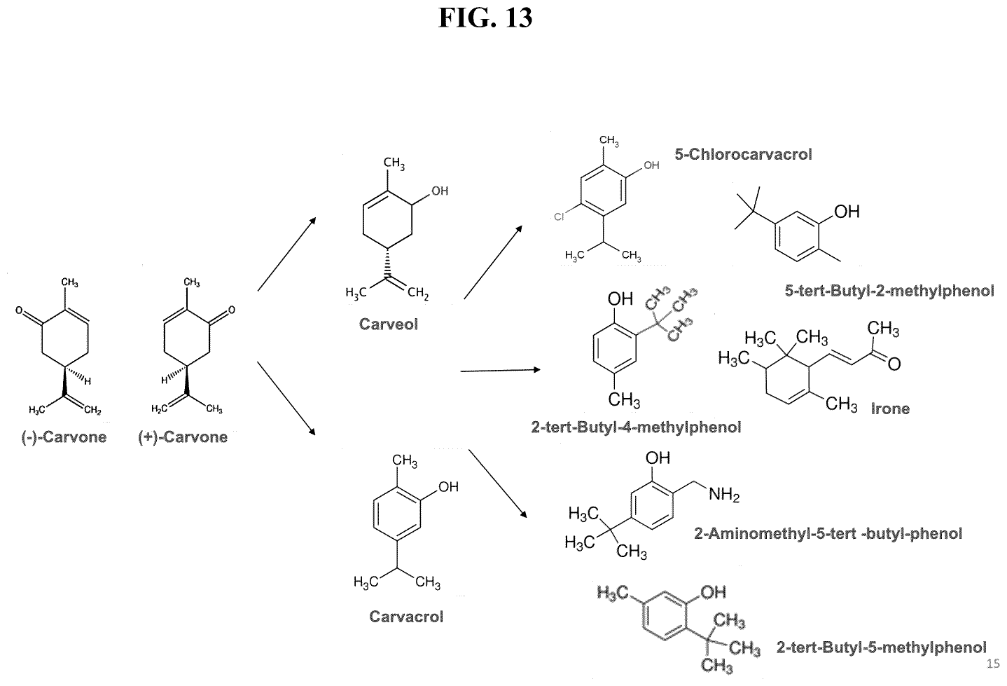

As blood transitions from steady laminar flow (S-flow) in healthy arteries to disturbed flow (D-flow) in aneurysmal arteries, platelets are subjected to external forces (see, e.g., FIG. 12 with hypothetical model). Biomechanical platelet activation is incompletely understood and is a potential mechanism behind antiplatelet medication resistance. While it was demonstrated that anti-platelet drugs suppress growth of abdominal aortic aneurysms (AAA) in patients, we revealed a certain degree of platelet reactivity persisted in spite of aspirin therapy. Transcriptomic profiling of platelets from patients with AAA revealed upregulation of a signal transduction pathway common to olfactory receptors (ORs), and this was explored as a mediator of AAA progression. Healthy platelets subjected to D-flow ex vivo, platelets from patients with AAA, and platelets in murine models of AAA demonstrated increased membrane olfactory receptor 2L13 (OR2L13) expression. A drug screen identified a molecule activating platelet OR2L13 which limited both biochemical and biomechanical platelet activation as well as AAA growth. This observation was further supported by selective deletion of the OR2L13 ortholog in a murine model of AAA that accelerated aortic aneurysm growth and rupture. These studies reveal that ORs regulate platelet activation in AAA and aneurysmal progression through platelet-derived mediators of aortic remodeling.

### Methods

### Odorant Screen

The control and RTP1s/OR2L13 reporter cells were used to perform odorant screening. Briefly, 10,000 reporter cells (control or RTP1s/OR2L13 expressing) were plated per well of 384 well cell culture plate in 10 ul of complete growth media without phenol red. Immediately after cell plating, 10 μl of 0-500 μM as an alpha screen odorant mix was added to each well in duplicate and gently vortexed. The screen was then repeated with odorant molecules individually in a dose-dependent manner. Forskolin (1 μM) and DMSO was added to one column of every assay plate to serve as a control. After the odorant mix addition, compounds from the Spectrum Collection (MS Discovery) were added to each well at ·1 μM concentration and incubated overnight at 37° C., 5% CO₂. The next day, 20 μL of One-Step Luciferase assay system (BPS Bioscience, San Diego, CA) was added to each plate and luciferase activity was read on a Gen5 plate reader. The ratio or OR2L13 cell line/vector cell line was used to indicate a positive response by way of a CRE reporter readout ratio of>1.0, then a dose response curve was constructed for lead compounds.

Ex vivo exposure of platelets to mechanical stress

Healthy human blood loaded with the fluorophore calcein green at room temperature (10 μM, pH 7.4 for 20 minutes), and incubated with odorant ligands for 30 minutes. Using a Cellix Microfluidics System (Cellix, Dublin, Ireland), blood was perfused over a collagen I pre-coated Vena8 Fluoro+biochip (15 μL at 150 μg/mL, humidified box overnight at 4° C., then washed with PBS) at 40 dyn/cm² using a Minis Nanopump. Image collection was conducted using an HC Plan Apo 20X/0.7NA lens mounted to a Leica DMI6000 inverted microscope and a dark chamber and a Hamamatsu ImagEM cooled charged coupled device camera. Platelets loaded with calcein green were used to determine thrombus size as % thrombus area at the end of three minutes using Image Pro plus software (Media Cybernetics, Rockville, Maryland, USA). Platelet exposure to S-flow (smooth surface) and D-flow (radial grooves) in vitro was made possible using a flow cone and plate system custom-manufactured by the University of Rochester department of engineering as described previously (19). The cone was rotated to provide S-flow shear at 15 dyn/cm². D-flow shear cannot be accurately determined, though the same rotational speed was used with S-flow and D-flow always run alongside no flow (static) conditions for internal consistency. Following exposure to S-flow or D-flow, platelets were imaged by confocal microscopy, surface marker proteins were determined quantitatively by flow cytometry, and platelet proteins were assessed by Western blotting.

### Experimental Animals

Mouse colony: Eight-week-old male wild-type C57/BL6 (Jackson laboratories, Bar Harbor, ME) and Fvb/Tac (Taconics Biosciences, Germantown, NY) mice were used in this study given that AAA is a disease affecting mostly males. The murine ortholog OR2L13 gene (olfr168) deletion mice were created in the University of Rochester functional genomic core using a CRISPR-Cas9 editing strategy with injection of the following guide RNA strands (Synthego Corporation): CAAGTGATTTCATTCTCTTA (SEQ ID NO:1) and GGGCCATGACAAGAGTCCTT (SEQ ID NO:2). Genotyping using primers spanning the predicted gene edit location were utilized and pups were further confirmed by Western blotting of isolated platelets using an OR2L13 antibody.

### AAA Surgery

We adapted the method demonstrated by Hu et al. to induce AAA with intraluminal thrombus (ILT) which is as close to the human phenotype of infrarenal AAA as possible (26). β-aminopropionitrile (BAPN) at 2 g/L is given in the mouse drinking water 2 days before the surgical procedure to inhibit lysl oxidase which would otherwise repair the damage to the aorta caused by topical elastase. To augment aneurysm growth and induce ILT as described (26), anti—TGFβ blocking antibody (250 μg/mouse) was injected i.p. three times weekly starting the day of surgery. Each mouse (20-30 g in weight) is given 1 mL subcutaneous physiologic saline before surgical incision to account for insensible volume losses with gut externalization. Surgical procedures were conducted on a surgical heating pad at 37 ° C. Mice were anesthetized with isoflurane anesthesia (3% by nosecone) using oxygen as the carrier. Buprenorphine (0.05 mg/Kg) is given 30 minutes prior to skin incision. The area of incision is cleaned with 70% ethanol and betadine three times, and then infiltrated gently with local anesthesia (bupivacaine 5 mg/Kg SQ immediately prior to incision). A midline abdominal incision is made through the skin. The rectus sheath was divided at the Linea Alba. The intestines are exteriorized with a Qtip to the left of mouse onto gauze soaked with saline above and below to prevent drying. Whatman paper 6 mm×9 mm slices were placed on top of aorta below the bifurcation of the renal arteries. A volume of 10 μL porcine pancreatic elastase, and dropped onto Whatman paper which is left in place for 5 minutes. For sham surgical animals, the procedure was identical, but heat-inactivated elastase was used (55° C., 10 minutes). Excess elastase was removed by adding 0.5 mL physiologic saline to the abdominal cavity, followed by an aspiration step. The mesentery was repositioned in the abdominal cavity. 6-0 vicryl interrupted sutures were used to close the peritoneum and 6-0 nylon was used to close skin using interrupted sutures. During recovery, a heated pad is placed under the mouse cage on one half only to assist in comfort while allowing the mouse the opportunity to move to a cooler area if desired. Hyperalgesia was assessed immediately after the procedure, and then twice daily for 48 hrs, and maintenance dose buprenorphine (0.05 mg/Kg) was given 8 hrs post-surgery, and then maintained at this dose for 48 hrs post-surgery. For the interventional study, Fvb/tac mice were injected with vehicle (DMSO) or (−) carvone starting on day 7 at the dose of 100 mg/Kg/day i.p. or given vehicle (water) or aspirin 30 mg/L ad libitum in drinking water starting on day 7. Blood draws were taken at time intervals no less than 1 week by the retro-orbital route into heparinized Tyrodes solution as we described previously (20, 27).

### Ultrasonography

Sonographic interrogation using pulsed wave Doppler allowed identification of the aorta. Color flow doppler interrogation was utilized to determine the presence of D-flow in aneurysmal segments and S-flow in the aorta of sham-operated animals. After carefully scanning the aorta of mice under general isoflurane anesthesia for the widest section of the aorta in short axis, color Doppler was used to identify the aorta and distinguish it from the vena cava. Pulsed wave Doppler interrogated the vessel to ensure the signal was arterial, and then M-mode was used through the widest section of the short axis of the aorta for enhanced temporal resolution. Aortic dimension at the maximum lumen diameter were taken at each time point as we described previously (4). The aortic edge-to-edge intima was measured using the Vevo2100 echocardiography system (VisualSonics, Toronto, Canada).

### Statistics

Data are presented as mean with standard error of the mean (SEM) unless otherwise stated. Normality and equal variance were firstly evaluated by the Shapiro—Wilk test. For normally-distributed data between two comparative groups, the Student's t-test was used. For non-parametric data, the Mann-Whitney U test was used. For Gaussian-distributed data in three or more groups, 1-way ANOVA followed by Bonferroni multiple comparisons test was used, otherwise the Kruskal—Wallis test followed by Dunn post-test was used. Significance was accepted as a P value<0.05. Analyses were conducted using GraphPad Prism 7 (GraphPad Software, Inc., La Jolla, CA).

### Results

Platelets from patients with AAA are highly reactive and enriched in activated WPs

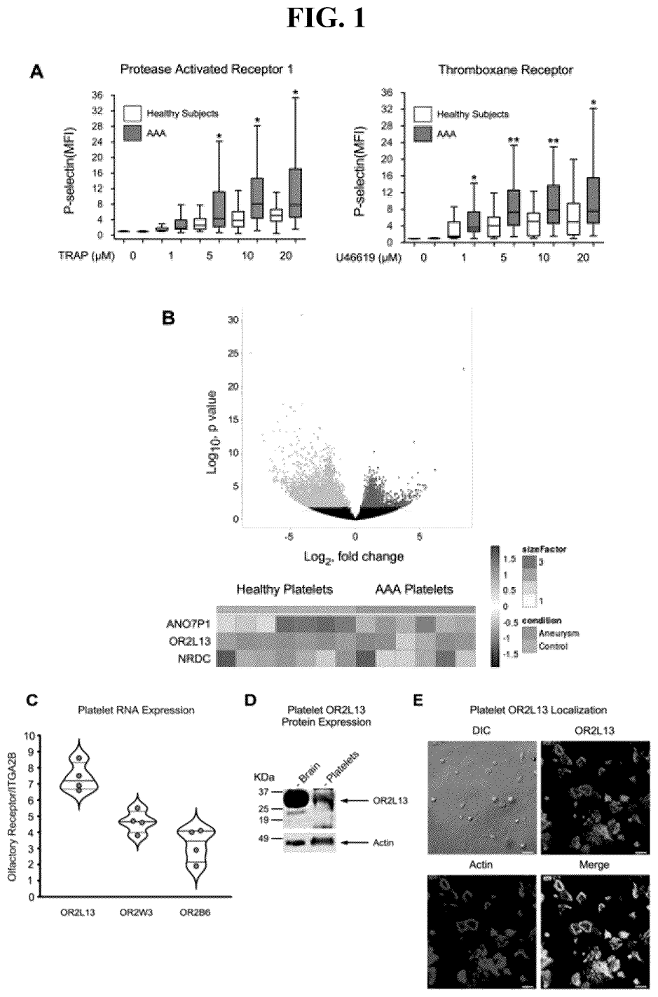

To determine whether platelets are more active in patients with AAA, we isolated washed platelets from patients or from healthy subjects and stimulated them with surface receptor agonists. Despite daily anti-platelet therapy for the majority of patients with AAA, platelet reactivity through both the thromboxane receptor and PAR1 was increased (FIG. 1A). Platelet hyperreactivity was not observed by stimulating the P2Y₁₂ receptor. Platelet surface PAR1 and thromboxane receptor density were not significantly different in patients with AAA compared with healthy control platelets. These findings suggest that circulating platelets in patients with AAA are phenotypically different.

### Olfactory Signaling Proteins are Increased in Platelets From AAA Patients

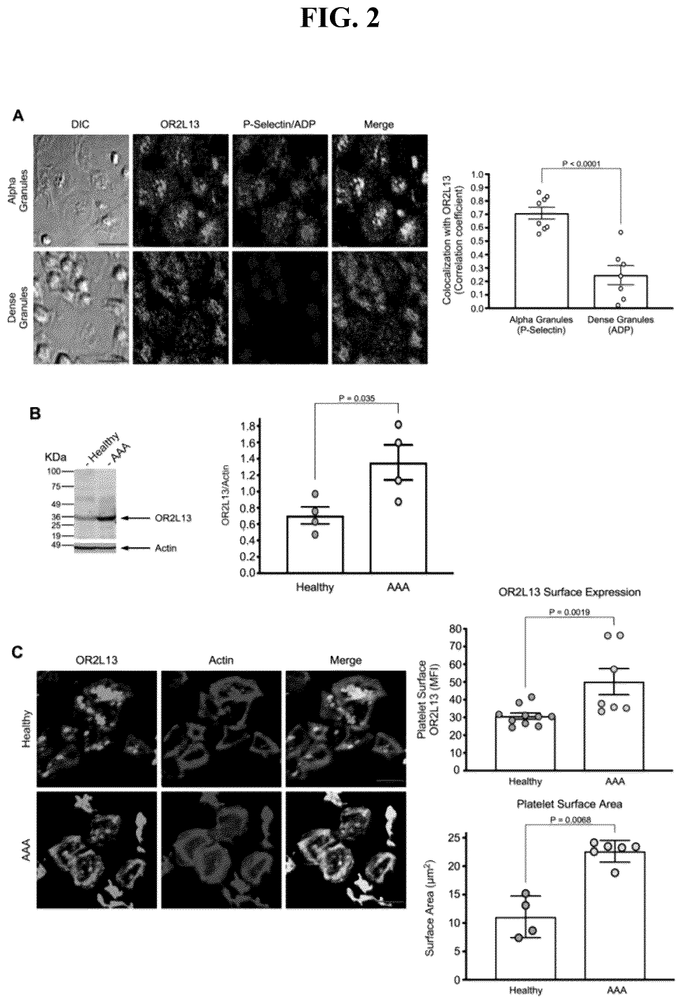

In order to determine platelet signaling pathways that may be altered in AAA, we isolated platelet RNA from healthy individuals and from patients with AAA then performed RNA sequencing (RNA-Seq). Differential expression of multiple transcripts were found between AAA and healthy control platelets, with the top two upregulated transcripts encoding OR2L13 and anoctamin 7 which are components of olfactory receptor signal transduction (FIG. 1B). More than 400 olfactory genes exist in the human genome (15). A functional role for platelet ORs has not previously been reported. We validated this primary observation in platelets using several complementary techniques. By employing RNA-seq in human CD34+, cord-blood derived, cultured megakaryocytes (platelet precursors), we examined which olfactory genes are endogenously expressed. We identified 15 non-truncated OR transcripts (including OR2L13) in human megakaryocytes. We validated the expression of only a subset of OR transcripts in twice washed CD45-depleted platelets from healthy adults by quantitative reverse transcriptase PCR (qRT-PCR) (FIG. 1C). Several OR pseudogenes with unclear function were also detected. Just three mature ORs were detected in adult platelets from every healthy subject tested: OR2L13, OR2W3, and OR2B6 (FIG. 1C). NRDC (Nardilysin Convertase), a required enzyme recently reported for platelet budding from megakaryocytes under D-flow conditions (16) was upregulated in platelets from patients with AAA by RNA-seq, and also found to be present in healthy platelets by qRT-PCR. Using human brain lysate as a positive control, healthy platelets were confirmed to express full-length OR2L13 protein by immunoblotting and OR2L13 membrane expression was observed by confocal microscopy (FIG. 1D-E). By co-staining platelet alpha granule and dense granule markers with OR2L13, co-expression of OR2L13 primarily with P-selectin suggested alpha granule storage and trafficking to the plasma membrane (FIG. 2A). Because OR2L13 was the only OR for which expression in platelets changed in AAA, we focused on understanding OR2L13 signal transduction. Protein expression of OR2L13 and Anoctamin 7 was increased in platelets from patients with AAA compared to healthy controls (FIG. 2B). Sex-dependent differences in platelet function was previously reported (7, 17, 18). However, we found platelet ORs were expressed similarly in healthy men and women. Membrane OR2L13 expression was increased in platelets from patients with AAA and AAA platelet spreading (surface area) on a fibrinogen matrix was also greater, further confirming enhanced platelet reactivity in AAA through the glycoprotein IIb/IIa (GPIIb/IIIa) receptor (FIG. 2C).

### OR2L13 is Biomechanically Sensitive

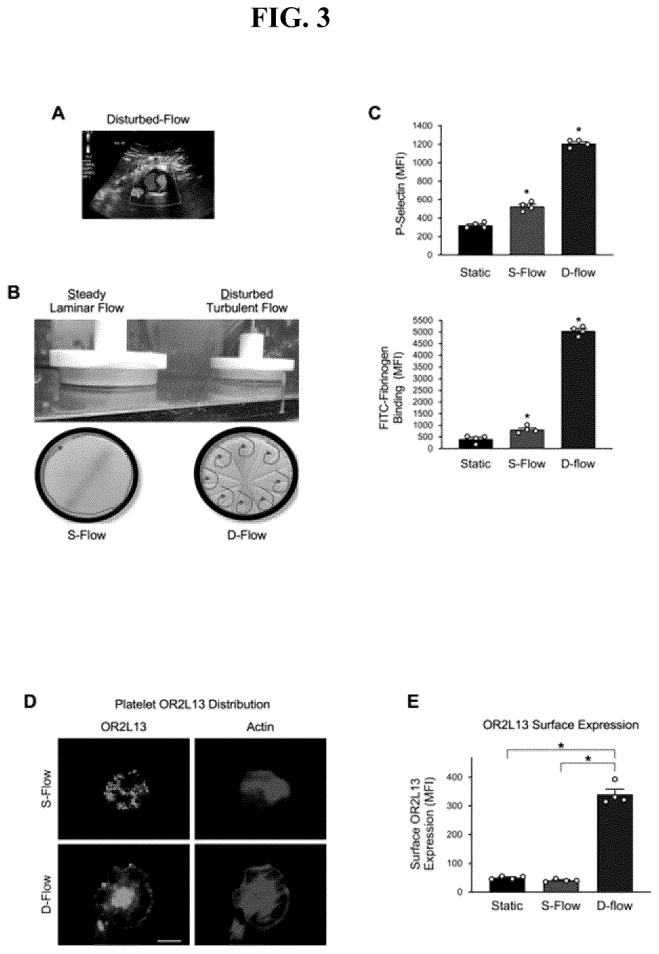

Patients with AAA have an aorta with an irregular shape, exposing circulating platelets to disturbed flow (D-flow) as noted by color spectral Doppler imaging (FIG. 3A). An ex vivo flow and cone system was used to recapitulate exposure of platelets to steady laminar flow (5-flow) and D-flow (19) (FIG. 3B). D-flow was an especially potent stimulus for biomechanical platelet activation (FIG. 3C), with OR2L13 converging in a central granulomere and on the membrane surface of permeabilized platelets (FIG. 3D). A marked increase in platelet membrane OR2L13 distribution was confirmed after non-permealized platelets were exposed to D-flow but not S-flow (FIG. 3E, flow cytometry) suggesting the nature of biomechanical platelet activation is a trigger for OR2L13 translocation. Since platelets can synthesize and degrade proteins in response to environmental stressors (20, 21), we assessed OR2L13 protein 25 expression following S-flow and D-flow exposure and found it to be similar to static conditions.

### OR2L13 Ligands and Platelet Function

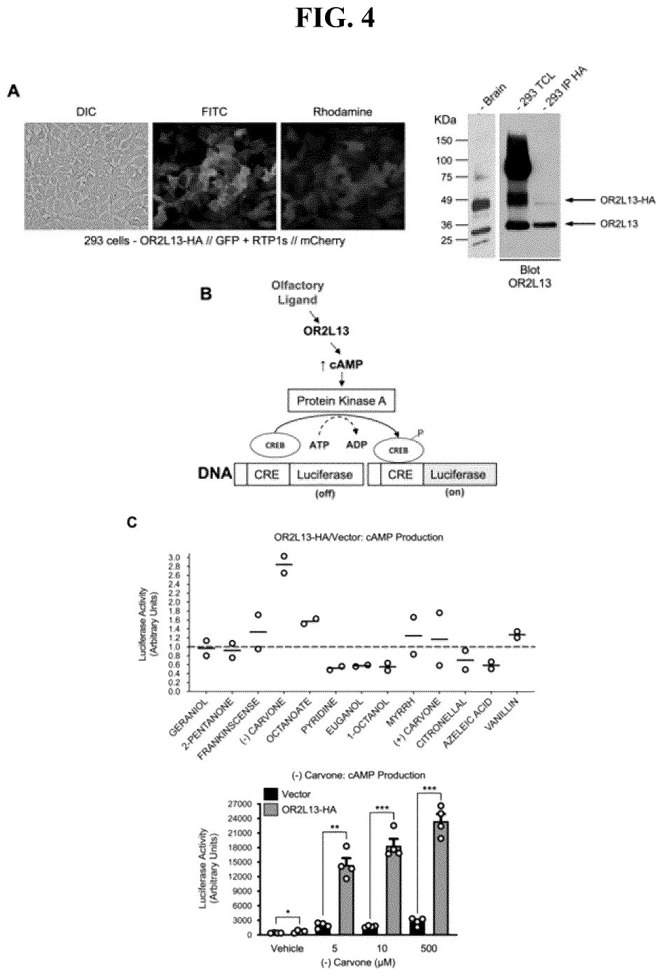

Following OR ligation by an external agonist, G_{olf} activates adenylyl cyclase (AC) to hydrolyze cyclic AMP (cAMP) from ATP. We developed a HEK293 reporter cell line to rapidly evaluate potential OR2L13 odorant ligands based on post-receptor cAMP production. Human OR2L13 cDNA was cloned in a bicistronic vector with a gene encoding the chaperone protein Receptor Transport Protein 1 subunit (RTP1s) to allow for efficient membrane localization (22). Lentivirus stably transduced OR2L13 and RTP1s in HEK293 cells with a cyclic AMP Response Element (CRE) in the 5′ position in-frame with luciferase as a biological reporter (FIG. 4A-B). Using cells expressing only the empty vector as a control, multiple potential olfactory receptor ligands were evaluated. The terpene derivative (−) carvone reproducibly activated OR2L13, generating endogenous cAMP in a dose-dependent manner compared with forskolin as a positive control for endogenous adenylyl cyclase (AC) activation (FIG. 4C).

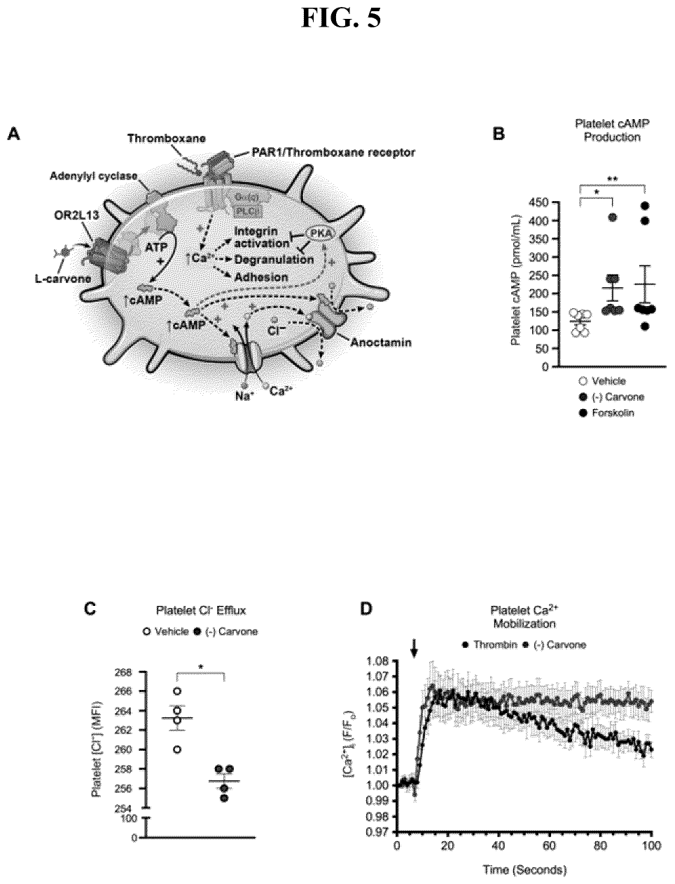

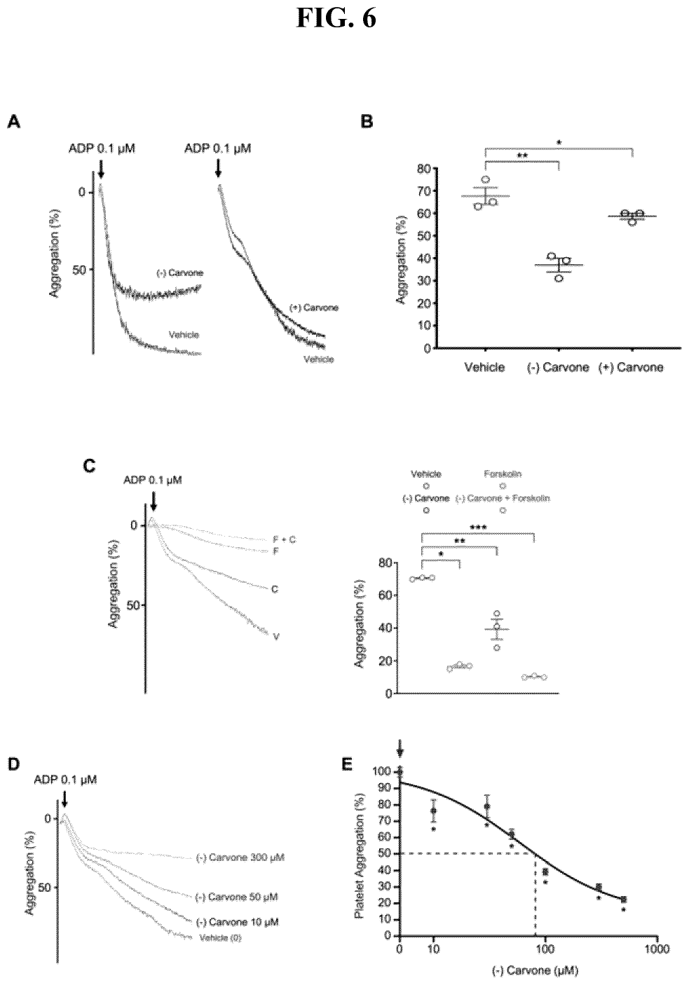

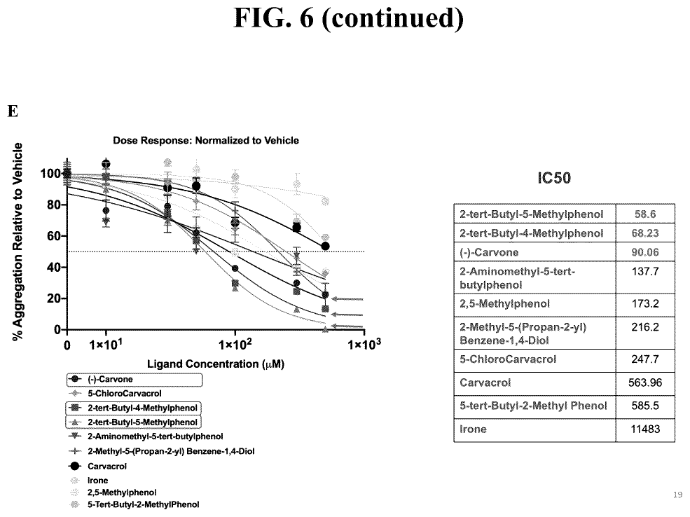

ORs and downstream anoctamin proteins are generally expressed in afferent olfactory neurons (23). ORs are GPCRs positively linked to AC to increase cAMP (24), triggering anoctamin 7 as a downstream calcium-sensitive chloride channel (25). If this signal transduction pathway is conserved in platelets, OR2L13 activation should generate cAMP in platelets coincident with changes in platelet Cl⁻- and Ca²⁺ flux (schematic, FIG. 5A). We indeed found (−) carvone generated cAMP in platelets and inhibited platelet aggregation compared to forskolin which generates cAMP in a receptor-independent manner (FIG. 5B). Consistent with the predicted signal transduction pathway in olfactory neurons, platelet OR2L13 activation by (−) carvone promoted platelet Cl⁻efflux and brief Ca² transients, presumably as downstream components of platelet post-receptor signal transduction (FIG. 5C-D). Carvone exists as levo (−) and dextro (+) enantiomers. We found the (−) enantiomer of carvone to have a more potent anti-platelet effect (FIG. 6A-B) and (−) carvone blunted ADP-induced platelet aggregation dose-dependently (FIG. 6C-E).

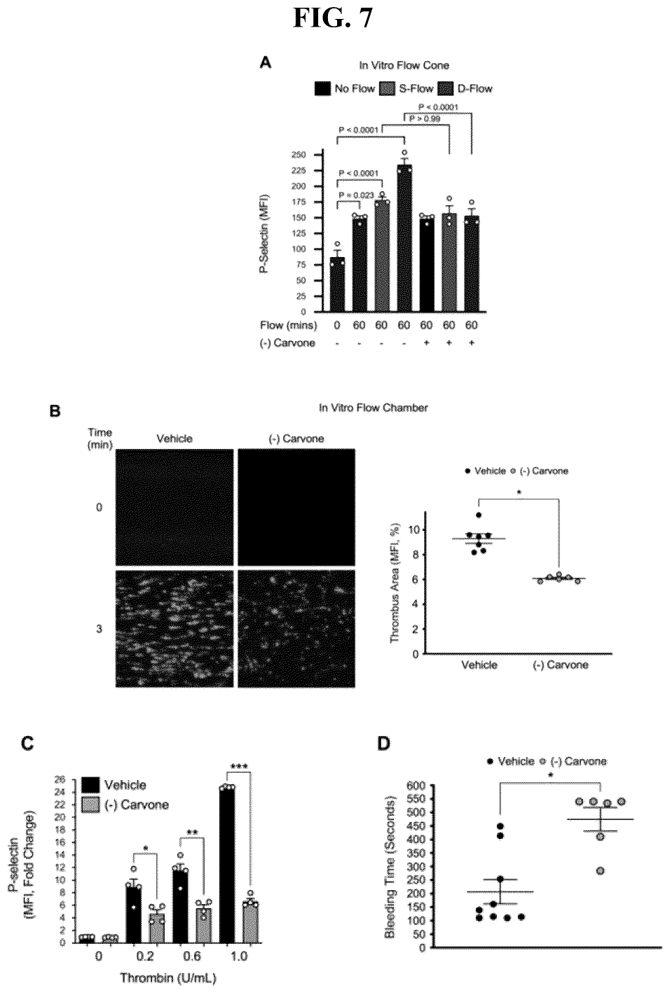

We next evaluated biomechanical platelet activation by S-flow and D-flow. Human platelets exposed to D-flow became activated and OR2L13 localizes to the membrane surface. We incubated healthy platelets with (−) carvone or control buffer before subjecting them to 5-flow or D-flow, observing (−) carvone attenuated only D-flow induced platelet activation (FIG. 7A). Utilizing another model of biomechanical platelet activation, vehicle or (−) carvone-treated human blood was passed through a collagen-coated microfluidic perfusion chamber to determine the rate of thrombus formation at high shear. Thrombosis in whole blood was attenuated by (−) carvone (FIG. 7B).

To ascertain whether exogenous (−) carvone in mice impacted platelet reactivity and thrombosis in vivo, FVB/tac mice were administered (−) carvone at a dose of 100 mg/Kg/day for three days by i.p. injection. Platelets were then isolated from mice and thrombin-stimulated ex vivo. Thrombin-induced platelet degranulation ex vivo was inhibited in platelets from (−) carvone-treated mice (FIG. 7C). Tail bleeding times in mice treated with (−) carvone were also prolonged (FIG. 7D) without affecting the circulating platelet number. This effect is consistent with adequate absorption and distribution of (−) carvone to attenuate platelet activation and thrombosis in vivo.

### Platelets Are Mechanically Activated in AAA Which Directs OR2L13 to the Membrane

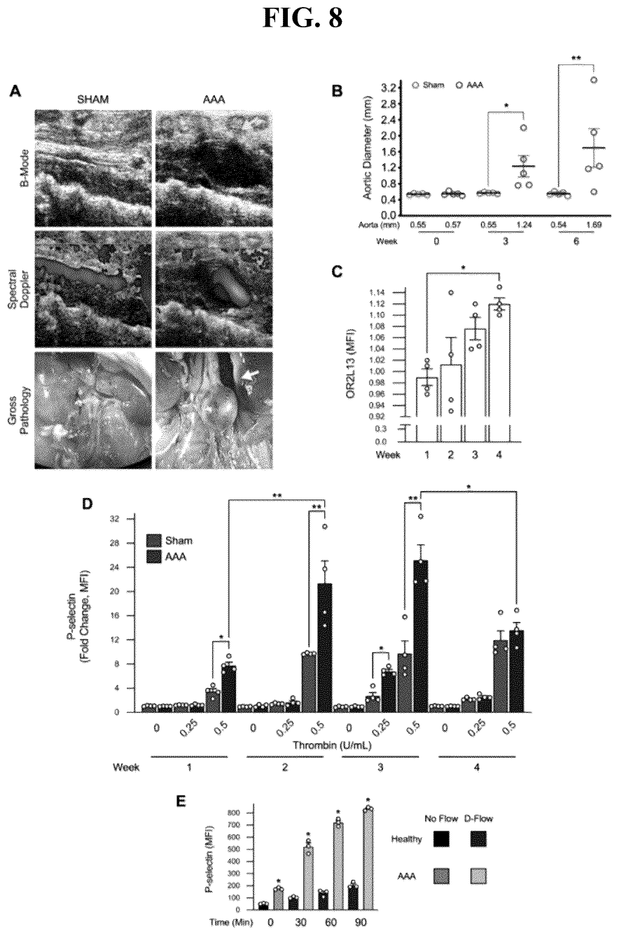

We recently reported that aspirin limits AAA growth in a large clinical population (10). Mice were injected three times weekly with an anti-TGFI3 antibody to induce aneurysmal growth and promote luminal thrombus formation. (26). Topical elastase was applied to the infrarenal aorta of mice to degrade elastin with the lysyl oxidase inhibitor BAPN administered ad libitum in the drinking water to prevent elastin crosslinking repair (26). To investigate a mechanism for this clinical observation, we explored the function of OR2L13 in a murine model of fast-growing AAA that develops luminal thrombus similar to human infrarenal AAA in which D-flow is observed in aneurysmal aortic regions (26). We confirmed this model of AAA exhibits D-flow in the aneurysmal segment and occasional luminal thrombus formation (FIG. 8A). After six weeks the aortic diameter increased 300% above baseline, consistent with severe AAA. Coincident with AAA growth, enhanced agonist-mediated platelet reactivity was observed, as well as increased surface OR2L13 expression in isolated platelets. Mice with AAA demonstrate enhanced platelet reactivity in which a D-flow environment pathologically appears compared to sham-operated mice with S-flow, reaching a plateau at 3 weeks and then dissipating at 4 weeks co-incident with increasing platelet surface OR2L13 expression (FIG. 8B-D). Furthermore, platelets isolated from patients with AAA were markedly sensitized to biomechanical activation ex vivo by D-flow compared to healthy platelets in a S-flow environment (FIG. 8E).

### OR2L13 Agonists Inhibit Platelet Activation and AAA Growth

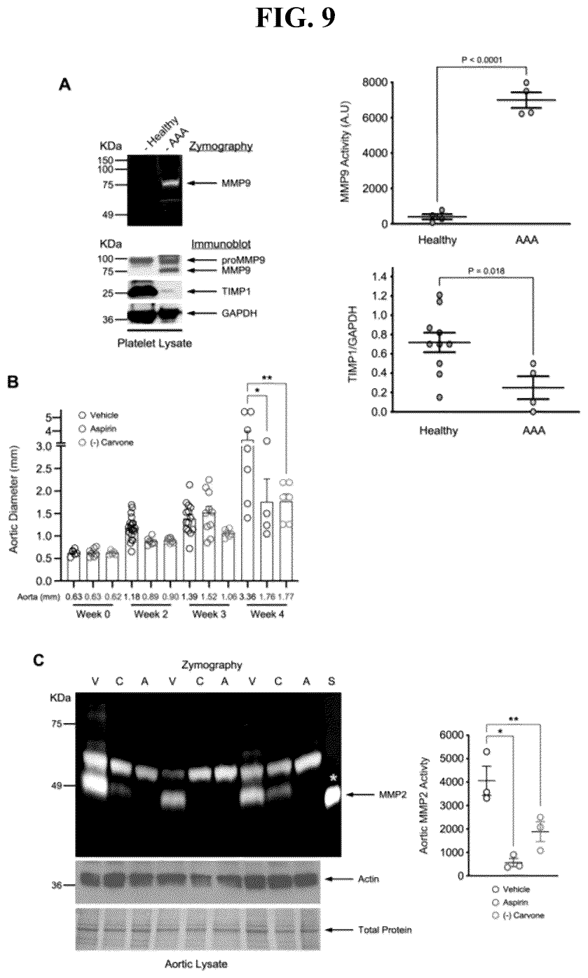

We previously reported that platelet matrix metalloproteinase (MMP) activity is increased in myocardial infarction (7, 27). Given that patients with AAA show less aortic growth and rupture when taking aspirin and growth and rupture are linked to MMP activity (10), we hypothesized that circulating platelets in patients with AAA may synthesize and secrete MMPs. Platelet MMP9 activity was enhanced and the MMP9 Tissue Inhibitor of MMP9 (TIMP1) was reduced in AAA compared to healthy conditions (FIG. 9A). Platelet MMP9 content was unchanged in healthy compared with AAA platelets. Aortic tissue as well as luminal thrombus from patients with infrarenal AAA compared to non-aneurysmal cadaveric aorta had increased expression and activity of MMP9. This observation suggests that platelets, which are a component of luminal thrombus, may contribute to aortic remodeling through MMPs. In our murine aneurysm model (26), (−) carvone treatment attenuated AAA growth similarly to aspirin, further suggesting anti-platelet therapeutics restrict aortic growth (FIG. 9). Consistent with the hypothesis that platelet-derived mediators regulate AAA growth, mice administered daily (−) carvone showed suppressed AAA growth coincident with decreased MMP2 activity in the aorta (FIG. 9C). The inhibitory effect of (−) carvone on aortic growth and MMP activation was similar to, but slightly more potent than, what was observed in aspirin-treated animals and suggests a platelet-mediated effect in regulating AAA progression (FIG. 9C).

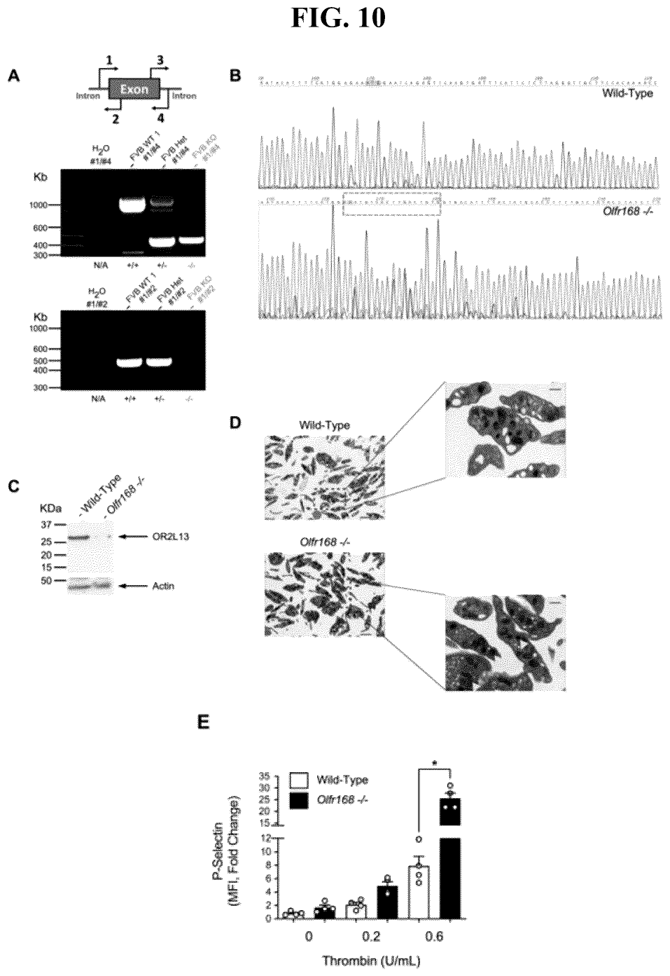

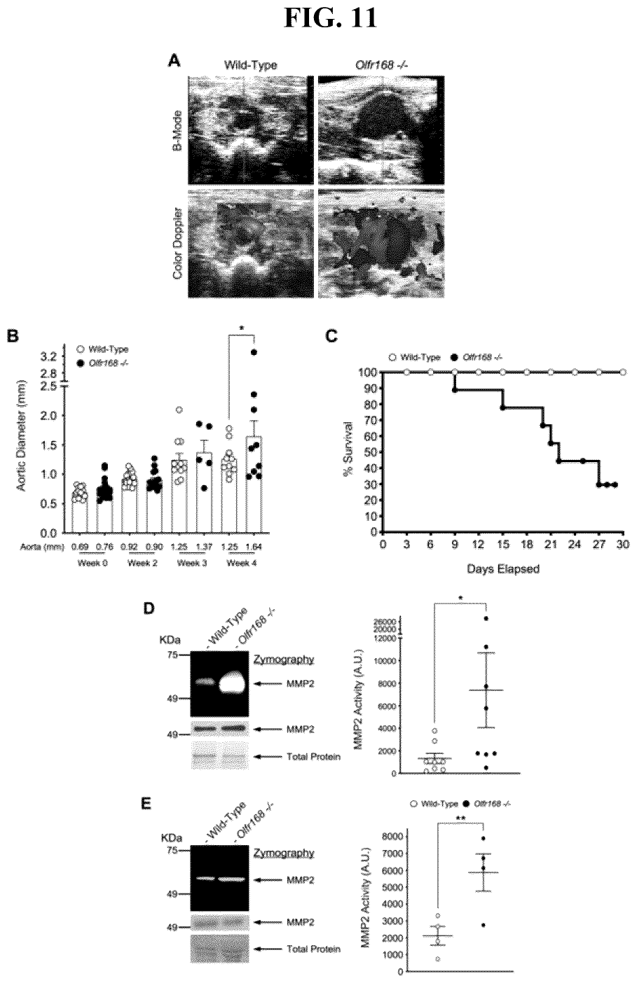

We examined platelets to determine the expression of the murine OR2L13 homologue olfr168 in various strains of mice and found the FVB/tac strain expressed OR2L13 at the greatest level compared to C57BL6/J mice that expressed little OR2L13. Using CRISPR-Cas9, we made an upstream edit in a unique sequence within the open reading frame of murine olfr168 resulting in gene deletion (FIG. 10A-B). Immunoblotting washed murine platelet lysate confirmed the absence of OR2L13 (olfr168^{-/-}) (FIG. 10C). Electron microscopic visualization of platelets showed enhanced granule content in olfr168^{-/-} mice coincident with enhanced platelet reactivity ex vivo compared with wild-type littermates (FIG. 10D-E). This further suggests olfr 168 may be an endogenous negative regulator of platelet function. Comparing wild-type with olfr168^{-/-} mice in the AAA model, the magnitude of D-flow observed in aneurysmal arterial segments, the rate of aortic growth, and incidence of aortic rupture all increased in mice with olfr168 deficiency (FIG. 11A-C). Finally, olfr168^{-/-} mice have augmented aortic MMP2 activity compared with WT mice (FIG. 11D) and isolated platelets from either wild-type mice treated with the olfr168 agonist (−) carvone or from olfr168^{-/-} mice have decreased and increased MMP2 activity compared to control mice, respectively (FIG. 11E). Deficiency of olfr168 did not affect platelet count, white blood cell (WBC) count, or hemoglobin concentration at baseline or following AAA induction. Together, while the present invention is not limited to any particular mechanism, and an understanding of the mechanism is not necessary to practice the invention, it is believe that these data imply that activated platelet OR2L13 is a protective mechanism to prevent excessive platelet reactivity in AAA and simultaneously silences platelet MMP2 activity. This may in part provide a mechanistic explanation for anti-platelet agents restricting AAA growth and rupture (4, 10).

This study shows a link between platelet reactivity and AAA progression in vivo, identifying functional ORs as novel signal transduction modules in platelets to serve as druggable targets. This discovery was made in aortic aneurysmal disease where platelet OR2L13 appears to be upregulated in response to biomechanical activation and, specifically, by external D-flow exposure. ORs are GPCRs which activate adenylyl cyclase to hydrolyze cAMP from ATP, a well-known second messenger that suppresses platelet reactivity (28). We identified the terpene (−) carvone, an active ingredient in spearmint, as a potent platelet OR2L13 agonist that generates cAMP endogenously in platelets. The same OR2L13 agonist suppresses thrombosis and platelet reactivity both ex vivo and in vivo. OR2L13 activators are therefore foundational to a new class of antiplatelet agents for thrombotic diseases.

In aneurysmal regions of the mouse and human aorta, D-flow and luminal thrombus are common observations and this may accelerate AAA growth (29-31). We show a similar fingerprint of MMP9 activity in platelets, aortic thrombus, and aorta in humans with advanced AAA. We identified that patients with AAA have markedly reduced platelet TIMP1 expression. TIMP1 is an inhibitor of MMP9 (32). Much like OR2L13 in this study, TIMP1 is stored in platelet alpha granules and secreted upon platelet activation (33). Platelet proteomic analysis previously demonstrated that TIMP1 expression inside platelets dramatically changes with aspirin pre-treatment (34).

OR signaling in non-olfactory tissue is a relatively new discovery but appears to regulate physiological processes including blood pressure, fat metabolism, and airway hyper-responsiveness (35-37). Hereditary anosmia (the inability to smell using functional ORs) coexists in patients with dysfunctional platelet activity—an observation made many years ago that was never explored (38). In addition, during the SARS-CoV-2 pandemic from 2019-2021, anosmia, presumably through inactivation of ORs is a common initial symptom of infection, and platelet activation as well as thrombosis are clear sequelae of this disease (39-41). These clinical observations raise the possibility that platelet ORs are mechanistic explanations for dysregulated platelet function in certain diseases. Of the several hundred ORs in the human genome, we determined only a few were expressed in the platelet precursor megakaryocyte, and even fewer at the protein level in mature adult platelets. Healthy platelets express reasonable quantities of OR2L13, OR2W2, and OR2B6. OR2L13 was the only platelet OR to change expression in AAA.

We show that OR2L13 co-localizes with P-selectin in platelets, suggesting alpha granule mobilization may be responsible for trafficking OR2L13 to the plasma membrane when platelets are activated by D-flow and further emphasizes OR2L13 has a protective role, attempting to attenuate activation of platelets persistently exposed to mechanical stimuli. We demonstrated using an OR2L13 ligand screen in vitro that (−) carvone is an OR2L13 agonist capable of increasing platelet cAMP which inhibits platelets, thrombosis, and AAA progression in vivo. The fact that OR2L13 activation increases cAMP production, chloride efflux, and transient calcium mobilization in platelets, similar to its native expression in olfactory afferent neurons, suggests a conserved signal transduction pathway (42-44). Over a decade ago, our group demonstrated platelets possess other functional signal transduction pathways common to neurons including the N-methyl-D-aspartate (NMDA) receptor, and the alpha-amino-3-hydroxy-5-methyl-4-isoxazolepropionic acid (AMPA) receptor (45). Recently, muscarinic acetylcholine receptors were found to regulate platelet reactivity and thrombosis (46). These previous studies and the current observations suggest that circulating platelets resemble neuronal synapses. In patients with AAA, we demonstrate that P2Y₁₂ receptor activation, which decreases platelet cAMP, is slightly inhibited, unlike PAR1 and the platelet thromboxane receptors which show enhanced activity in AAA. Patients with AAA have increased platelet OR2L13 expression, a receptor pathway that increases cAMP, suggesting development of a protective mechanism involving enhanced adenylyl cyclase activity and cAMP production in circulating platelets of patients with AAA. Several platelet surface GPCRs positively couple to adenylyl cyclase to generate cAMP (47). OR2L13 agonists could be alternative antiplatelet agents for patients with cardiovascular disease in whom P2Y₁₂ receptor antagonists are ineffective (48, 49). In a recent clinical trial in patients with small infrarenal aortic aneurysms, a P2Y₁₂ receptor antagonist did not impair aneurysmal growth when administered in a randomized, blinded manner (14). The P2Y12 receptor, therefore, may play a smaller role in platelet-mediated AAA growth, as suggested by our data. Alternatively, medical intervention for small aortic aneurysms or the time of treatment during AAA development in the prior report may generate an insufficient magnitude of D-flow to activate platelets.

We discovered that platelets from patients with AAA are biomechanically activated in spite of aspirin therapy but can be suppressed by the OR2L13 agonist (−) carvone. Parenteral administration of (−) carvone in mice inhibits the increase in platelet reactivity and suppresses AAA growth in vivo.

In summary, this investigation is the first to identify functional platelet olfactory receptors and show surface OR2L13 stimulation suppresses platelet reactivity in humans and in mice. When platelet OR2L13 is activated during AAA development by an exogenous, activating ligand, the biological consequence is suppression of platelet and aortic MMP activity, limiting aortic aneurysm progression. Conversely, OR2L13 deficiency augments platelet and aortic MMP activity, promoting aneurysmal growth and aortic rupture.

### REFERENCRES

1. Howard et al. Population-based study of incidence and outcome of acute aortic dissection and premorbid risk factor control: 10-year results from the Oxford Vascular Study. Circulation. 2013;127(20):2031-7.

2. Sakalihasan et al., Activated forms of MMP2 and MMP9 in abdominal aortic aneurysms. J Vasc Surg. 1996;24(1):127-33.

3. Marcos et al. Effect of antiplatelet therapy on aneurysmal sac expansion associated with type II endoleaks after endovascular aneurysm repair. J Vasc Surg. 2017;66(2):396-403.

4. Owens et al. Platelet Inhibitors Reduce Rupture in a Mouse Model of Established Abdominal Aortic Aneurysm. Arterioscler Thromb Vasc Biol. 2015;35(9):2032-41.

5. Robless et al., Increased platelet aggregation and activation in peripheral arterial disease. Eur J Vasc Endovasc Surg. 2003;25(1):16-22.

6. Hansen et al., Mechanical platelet activation potential in abdominal aortic aneurysms. J Biomech Eng. 2015;137(4):041005.

7. Schmidt et al. The platelet phenotype in patients with ST-segment elevation myocardial infarction is different from non-ST-segment elevation myocardial infarction. Transl Res. 2018;195:1-12.

8. Cameron et al., A Novel Anti-Inflammatory Effect for High Density Lipoprotein. PLoS One. 2015;10(12):e0144372.

9. Nishiguchi et al. Local Matrix Metalloproteinase 9 Level Determines Early Clinical Presentation of ST-Segment-Elevation Myocardial Infarction. Arterioscler Thromb Vasc Biol. 2016;36(12):2460-7.

10. Elbadawi et al. Antiplatelet Medications Protect Against Aortic Dissection and Rupture in Patients With Abdominal Aortic Aneurysms. J Am Coll Cardiol. 2020;75(13):1609-10.

11. Chen et al. An integrin alphaIIbbeta3 intermediate affinity state mediates biomechanical platelet aggregation. Nat Mater. 2019;18(7):760-9.

12. Bhagavan et al., Strongly Coupled Morphological Features of Aortic Aneurysms Drive Intraluminal Thrombus. Sci Rep. 2018;8(1):13273.

13. Haller et al. Intraluminal thrombus is associated with early rupture of abdominal aortic aneurysm. J Vasc Surg. 2018;67(4):1051-8 el.

14. Wanhainen et al. The effect of ticagrelor on growth of small abdominal aortic aneurysms-a randomized controlled trial. Cardiovasc Res. 2020;116(2):450-6.

15. Malnic et al., The human olfactory receptor gene family. Proc Natl Acad Sci U.S. A. 2004;101(8):2584-9.

16. Ito et al. Turbulence Activates Platelet Biogenesis to Enable Clinical Scale Ex Vivo Production. Cell. 2018;174(3):636-48 e18.

17. Soo Kim B, Auerbach D S, Sadhra H, Godwin M, Bhandari R, Ling F S, et al. Sex-Specific Platelet Activation Through Protease-Activated Receptors Reverses in Myocardial Infarction. Arterioscler Thromb Vasc Biol. 2021;41(1):390-400.

18. Godwin M D, Aggarwal A, Hilt Z, Shah S, Gorski J, and Cameron S J. Sex-Dependent Effect of Platelet Nitric Oxide: Production and Platelet Reactivity in Healthy Individuals. JACC Basic Transl Sci. 2022;7(1):14-25.

19. Heo K S, Lee H, Nigro P, Thomas T, Le N T, Chang E, et al. PKCzeta mediates disturbed flow-induced endothelial apoptosis via p53 SUMOylation. J Cell Biol. 2011;193(5):867-84.

20. Cameron S J, Mix D S, Ture S K, Schmidt R A, Mohan A, Pariser D, et al. Hypoxia and Ischemia Promote a Maladaptive Platelet Phenotype. Arterioscler Thromb Vasc Biol. 2018.

21. Weyrich A S, Schwertz H, Kraiss L W, and Zimmerman G A. Protein synthesis by platelets: historical and new perspectives. J Thromb Haemost. 2009;7(2):241-6.

22. Wu L, Pan Y, Chen G Q, Matsunami H, and Zhuang H. Receptor-transporting protein 1 short (RTP1S) mediates translocation and activation of odorant receptors by acting through multiple steps. J Biol Chem. 2012;287(26):22287-94.

23. Zak J D, Grimaud J, Li R C, Lin C C, and Murthy V N. Calcium-activated chloride channels clamp odor-evoked spike activity in olfactory receptor neurons. Sci Rep. 2018;8(1):10600.

24. Nakashima N, Nakashima K, Taura A, Takaku-Nakashima A, Ohmori H, and Takano M. Olfactory marker protein directly buffers cAMP to avoid depolarization-induced silencing of olfactory receptor neurons. Nat Commun. 2020;11(1):2188.

25. Duran C, and Hartzell H C. Physiological roles and diseases of Tmem16/Anoctamin proteins: are they all chloride channels? Acta Pharmacol Sin. 2011;32(6):685-92.

26. Lareyre F, Clement M, Raffort J, Pohlod S, Patel M, Esposito B, et al. TGFbeta (Transforming Growth Factor-beta) Blockade Induces a Human-Like Disease in a Nondissecting Mouse Model of Abdominal Aortic Aneurysm. Arterioscler Thromb Vasc Biol. 2017;37(11):2171-81.

27. Cameron S J, Ture S K, Mickelsen D, Chakrabarti E, Modjeski K L, McNitt S, et al. Platelet Extracellular Regulated Protein Kinase 5 Is a Redox Switch and Triggers Maladaptive Platelet Responses and Myocardial Infarct Expansion. Circulation. 2015;132(1):47-58.

28. Hollopeter G, Jantzen H M, Vincent D, Li G, England L, Ramakrishnan V, et al. Identification of the platelet ADP receptor targeted by antithrombotic drugs. Nature. 2001;409(6817):202-7.

29. Ding Y, Li X, Zhou M, Cai L, Tang H, Xie T, et al. Factor Xa inhibitor rivaroxaban suppresses experimental abdominal aortic aneurysm progression via attenuating aortic inflammation. Vascul Pharmacol. 2020:106818.

30. Cameron S J, Russell H M, and Owens A P, 3rd. Antithrombotic therapy in abdominal aortic aneurysm: beneficial or detrimental? Blood. 2018;132(25):2619-28.

31. Lindquist Liljeqvist M, Hultgren R, Bergman O, Villard C, Kronqvist M, Eriksson P, et al. Tunica-Specific Transcriptome of Abdominal Aortic Aneurysm and the Effect of Intraluminal Thrombus, Smoking, and Diameter Growth Rate. Arterioscler Thromb Vasc Biol. 2020;40(11):2700-13.

32. Visse R, and Nagase H. Matrix metalloproteinases and tissue inhibitors of metalloproteinases: structure, function, and biochemistry. Circ Res. 2003;92(8):827-39.

33. Villeneuve J, Block A, Le Bousse-Kerdiles M C, Lepreux S, Nurden P, Ripoche J, et al. Tissue inhibitors of matrix metalloproteinases in platelets and megakaryocytes: a novel organization for these secreted proteins. Exp Hematol. 2009;37(7):849-56.

34. Shah P, Yang W, Sun S, Pasay J, Faraday N, and Zhang H. Platelet glycoproteins associated with aspirin-treatment upon platelet activation. Proteomics. 2017;17(6).

35. Pluznick J L, Protzko R J, Gevorgyan H, Peterlin Z, Sipos A, Han J, et al. Olfactory receptor responding to gut microbiota-derived signals plays a role in renin secretion and blood pressure regulation. Proc Natl Acad Sci USA. 2013;110(11):4410-5.

36. Wu C, Hwang S H, Jia Y, Choi J, Kim Y J, Choi D, et al. Olfactory receptor 544 reduces adiposity by steering fuel preference toward fats. J Clin Invest. 2017;127(11):4118-23.

37. Huang J, Lam H, Koziol-White C, Limjunyawong N, Kim D, Kim N, et al. The odorant receptor OR2W3 on airway smooth muscle evokes bronchodilation via a cooperative chemosensory tradeoff between TMEM16A and CFTR. Proc Natl Acad Sci USA. 2020;117(45):28485-95.

38. Pawlowitzki I H, Diekstall P, Miny P, and Balleisen L. Abnormal platelet function in Kallmann syndrome. Lancet. 1986;2(8499):166.

39. Hottz E D, Azevedo-Quintanilha I G, Palhinha L, Teixeira L, Barreto E A, Pao CRR, et al. Platelet activation and platelet-monocyte aggregate formation trigger tissue factor expression in patients with severe COVID-19. Blood. 2020;136(11):1330-41.

40. Manne B K, Denorme F, Middleton E A, Portier I, Rowley J W, Stubben C, et al.

Platelet gene expression and function in patients with COVID-19. Blood. 2020;136(11):1317-29.

41. Lechien J R, Chiesa-Estomba C M, Beckers E, Mustin V, Ducarme M, Journe F, et al. Prevalence and 6-month recovery of olfactory dysfunction: a multicentre study of 1363 COVID-19 patients. J Intern Med. 2021.

42. Wright R H. Odour and molecular vibration. Nature. 1966;209(5023):571-2.

43. Wright R H. Odor and molecular vibration: response to nitrobenzene-d5 of honey bees (Apis mellifera L.) conditioned with nitrobenzene. Experientia. 1975;31(5):530.

44. Maniati K, Haralambous K J, Turin L, and Skoulakis E M C. Vibrational Detection of Odorant Functional Groups by Drosophila melanogaster. eNeuro. 2017;4(5).

45. Morrell C N, Sun H, Ikeda M, Beique J C, Swaim A M, Mason E, et al. Glutamate mediates platelet activation through the AMPA receptor. J Exp Med. 2008;205(3):575-84.

46. Bennett J A, Ture S K, Schmidt R A, Mastrangelo M A, Cameron S J, Terry L E, et al. Acetylcholine Inhibits Platelet Activation. J Pharmacol Exp Ther. 2019;369(2):182-7.

47. Noe L, Peeters K, Izzi B, Van Geet C, and Freson K. Regulators of platelet cAMP levels: clinical and therapeutic implications. Curr Med Chem. 2010;17(26):2897-905.

48. Correll M, Johnson C K, Ferrari G, Brizzio M, Mak A W, Quackenbush J, et al. Mutational analysis clopidogrel resistance and platelet function in patients scheduled for coronary artery bypass grafting. Genomics. 2013;101(6):313-7.

49. Strisciuglio et al. Impact of genetic polymorphisms on platelet function and response to anti platelet drugs. Cardiovasc Diagn Ther. 2018;8(5):610-20.

All publications and patents mentioned in the specification and/or listed below are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope described herein.

1. A method of treating a subject with, or elevated risk for, a cardiovascular disease (CVD) involving dysregulated platelet activation comprising:
treating a subject with a composition comprising an OR2L13 activating agent;
wherein said subject has, or is at elevated risk for, a CVD involving dysregulated platelet activation; and
optionally, wherein said OR2L13 activating agent comprises: 2-tert-Butyl-4-Methylphenol or 2-tert-Butyl-5-Methylphenol.

2. The method of claim 1, wherein said OR2L13 activating agent binds OR2L13 on platelets in said subject.

3. The method of claim 1, wherein said OR2L13 activating agent reduces platelet activation in said subject.

4. The method of claim 1, wherein said CVD comprises abdominal aortic aneurysm (AAA).

5. The method of claim 1, wherein said CVD comprises thrombosis.

6. The method of claim 1, wherein said CVD is selected from the group consisting of: myocardial infarction, stroke, coronary artery disease, angina, heart failure, heart valve disease, and heart attack.

7. The method of claim 1, wherein said OR2L13 activating agent comprises Carvone.

8. The method of claim 7, wherein said Carvone comprises (−) Carvone.

9. The method of claim 1, wherein said OR2L13 activating agent is selected from the group consisting of: 2-tert-Butyl-5-Methylphenol; 2-tert-Butyl-4-Methylphenol; (−)-Carvone; 2-Aminomethyl-5-tert-butylphenol; 2,5-Methylphenol; 2-Methyl-5-(Propan-2-yl) Benzene-1,4-Diol; 5-ChloroCarvacrol; Carvacrol; 5-tert-Butyl-2-Methyl Phenol; and Irone.

10. The method of claim 1, wherein said subject is a human.

11. The method of claim 1, wherein treating comprises administering said composition to said subject orally.

12. The method of claim 1, wherein treating comprises administering said composition to said subject via an aerosol.

13. The method of claim 1, wherein said treating comprises administering said composition to said subject intravenously.

14. The method of claim 1, wherein said treating comprises providing said composition to said subject such that they administer the composition to themselves.

15. The method of claim 1, further comprising, prior to said treating, the step of testing a blood or plasma sample from said subject to determine if platelets in said sample are activated.

16. The method of claim 1, further comprising, after said treating, the step of testing a blood or plasma sample from said subject to determine if platelets in said sample are activated.

17. The method of claim 1, wherein said subject has a CVD involving dysregulated platelet activation.

18. The method of claim 1, wherein said subject is at elevated risk for a CVD involving dysregulated platelet activation.

19. The method of claim 18, wherein said subject has been diagnosed as having an elevated risk for a CVD involving dysregulated platelet activation.

20. A composition comprising:
a) a blood or plasma sample from a subject that has, or is at elevated risk for, a CVD involving dysregulated platelet activation; and
b) a composition comprising an OR2L13 activating agent, and optionally, wherein said OR2L13 activating agent comprises: 2-tert-Butyl-4-Methylphenol or 2-tert-Butyl-5-Methylphenol.

21-24. (canceled)


SEQUENCE LISTING (4 sequences)

SEQ ID NO: 1 - RNA, 20 nt, synthetic construct
  source 1..20: mol_type = other RNA
caagtgatttcattctctta

SEQ ID NO: 2 - RNA, 20 nt, synthetic construct
  source 1..20: mol_type = other RNA
gggccatgacaagagtcctt

SEQ ID NO: 3 - DNA, 72 bp, Mus musculus
  source 1..72: mol_type = genomic DNA
aatacattttcatggagaaatggaatcagagttcaagtgatttcattctcttagggttgcttccacaaaacc

SEQ ID NO: 4 - DNA, 74 bp, Mus musculus
  source 1..74: mol_type = other DNA
aatacattttcatggaaatgtcacttgactgtggtgacattttactatgcaccttttgtctatacctatcttcg